(19) United States
(12) Patent Application Publication
(10) Pub. No.: US 2005/0131507 A1
(43) Pub. Date: Jun. 16, 2005

(54) Lead having reduced lead body size

(76) Inventor: Gregory L. Sundberg, Stillwater, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/734,088

(22) Filed: Dec. 11, 2003

(51) Int. Cl. A61N 1/05

(52) U.S. Cl. 607/122

(57) ABSTRACT

An implantable device such as a lead with a lead body is adapted for implantation on or about the heart and for connection to a system for monitoring or stimulating cardiac activity. The implantable device includes one or more fillers disposed within the lead body resulting in a lead body having a reduced lead body size.

### TECHNICAL FIELD

Leads for conducting electrical signals to and from the heart, and more particularly, leads having reduced lead body size.

### TECHNICAL BACKGROUND

Pacemaker leads represent the electrical link between the pulse generator and the heart tissue, which is to be excited and/or sensed. These pacemaker leads include single or multiconductors that are connected to an electrode in an electrode assembly at an intermediate portion or distal end of a pacing lead. A connector is included at the proximal end to form the electrical connection with the pacemaker.

To implant the lead within the patient, the lead is often fed intravenously toward the heart. The lead may be implanted within or travel through complex or tortuous vasculature. The lead may also need to travel through vasculature having increasingly smaller diameters.

There is a need for a lead having a lead body with reduced outer diameter. In addition, there is a need for a lead with a lead body that minimizes trauma to the tissue.

### SUMMARY

An implantable lead includes a lead body, at least one electrode disposed along the lead body, at least one conductor electrically coupled with the at least one electrode, and one or more fillers disposed within the lead body, the one or more fillers are disposed adjacent to the at least one conductor. In one option, the conductor is not coupled with the electrode. In another option, each filler fills less than about 50% of the cross-sectional area of the lead body.

Several options for the lead are as follows. In one option, the fillers include compression features associated therewith, such as, but not limited to, compression waves disposed on an inner perimeter of the one or more fillers. The fillers are, in one option, C-shaped. In another option, two fillers are disposed within the lead body, each filler has a first end and a second end, and a first conductor is disposed between two first ends of the two fillers, and a second conductor is disposed between two second ends of the two fillers. The lead further optionally includes a coiled conductor with a lumen, the coiled conductor is disposed within the lead body, and a coil conductor longitudinal axis is offset from a lead body longitudinal axis.

A method is provided including disposing two or more conductors within an insulative lead body, where the two or more conductors include a coiled conductor and at least one cable conductor. The method further includes electrically coupling an electrode with at least one conductor, and disposing one or more fillers within the lead body without coupling the conductors with the one or more fillers.

Several options for the method are as follows. For example, in one option, disposing the coiled conductor within the lead body includes disposing the coiled conductor at a location offset from a longitudinal axis of the lead body. In another option, disposing one or more fillers includes disposing two or more fillers on opposite sides of the coiled conductor. The method further includes, in another option, insulating the coiled conductor and the at least one cable conductor. In yet another option, disposing one or more fillers within the lead body includes disposing a C-shaped filler within the lead body, the C-shape having an inner perimeter portion, and disposing the coiled conductor within the inner perimeter portion. In another embodiment, disposing one or more fillers within the lead body includes disposing one or more fillers with compression features within the lead body.

These and other embodiments, aspects, advantages, and features will be set forth in part in the description which follows, and in part will become apparent to those skilled in the art by reference to the following description and referenced drawings or by practice thereof. The aspects, advantages, and features are realized and attained by means of the instrumentalities, procedures, and combinations particularly pointed out in the appended claims and their equivalents.

### BRIEF DESCRIPTION OF THE DRAWINGS

FIG. 1 is a diagram illustrating a lead system constructed in accordance with one embodiment.

FIG. 2 is a cross-sectional view of a portion of a lead taken along A-A constructed in accordance with one embodiment.

FIG. 3 is a cross-sectional view of a portion of a lead taken along A-A constructed in accordance with one embodiment.

FIG. 4 is a cross-sectional view of a portion of a lead taken along A-A constructed in accordance with one embodiment.

### DESCRIPTION OF THE EMBODIMENTS

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the spirit and scope of the present invention. Therefore, the following detailed description is not to be taken in a limiting sense, and the scope is defined by the appended claims.

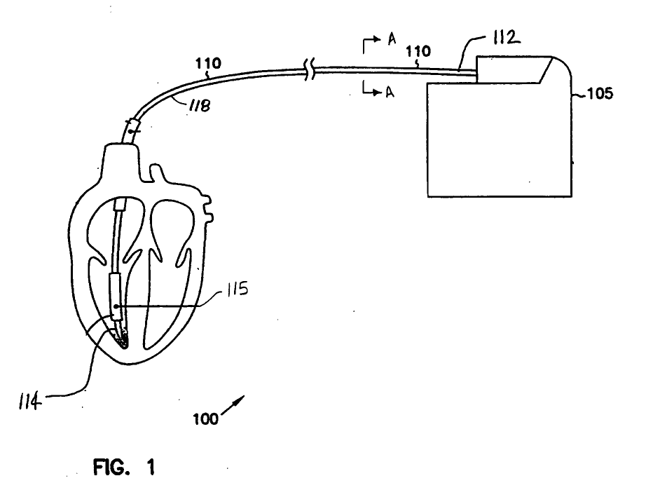

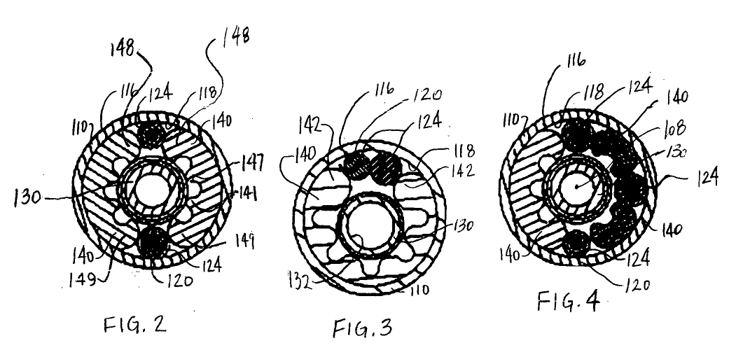

An implantable device 100, such as a lead for use with an electrical stimulator 105, is illustrated in FIG. 1. The implantable device 100 includes a lead body 110, and at least one elongate conductor 120 (FIGS. 2-4) contained within the lead body 110. The lead body 110 extends from a proximal end 112 to a distal end 114. The proximal end 112 of the lead is electrically coupled with the electrical stimulator 105, for example, with a connector.

In one option, the electrical stimulator 105 is a pulse sensor and generator that contains electronics to sense various electrical signals of the heart and also produce current pulses for delivery to the heart. The pulse sensor and generator also contains electronics and software necessary to detect certain types of arrhythmias and to correct for them.

The implantable device 100 further includes, in one option, one or more electrodes 115. The one or more electrodes 115 are each electrically coupled with the at least one conductor 120 (FIGS. 2-4). The electrode 115 allows for electrical signals to be delivered to the tissue from the electrical stimulator 105. The implantable device 100 further includes, in one option, features to allow the lead body to be fixated within a patient. For example, in one option, the lead body includes passive fixation features, such as one or more tines. In another option, the lead body includes an active fixation assembly, such as a fixation helix.

Referring to FIG. 2, the lead body 110 is a tubular body that is defined in part by an outer surface 116 and an inner surface 118. The inner surface 118 forms an outer perimeter defining a surface area at the cross-section for the lead body 110. Disposed within the lead body 110 is at least one conductor 120. In one option, a plurality of conductors is disposed therein. For example, one or more cable conductors 124 are disposed within the lead body 110. In one option, as illustrated in FIGS. 2 and 3, two cable conductors 124 are disposed within the lead body 110. Optionally, the one or more cable conductors 124 are disposed adjacent to one another, as illustrated in FIG. 3. One or more insulative coatings or sleeves disposed on the cable conductors 124, such as, but not limited to, PTFE, EFTE, or polyurethane, that allows for the cable conductors 124 to be disposed directly adjacent to one another. The coating, or flexible sleeve is capable of providing electrical isolation, and abrasion resistance. In another option, the two cable conductors 124 are disposed on opposite sides of a coiled conductor (discussed below) as illustrated in FIG. 2. In yet another option, three or more cable conductors 124 are disposed within the lead body 110, as illustrated in FIG. 4.

In another example, a coiled conductor 130 is disposed within the lead body 110, for example, in addition to the cable conductors 124. The coiled conductor 130 has, in one option, individual filars that are coated or otherwise covered with an insulative material. For example, a cross-section of the individual coiled filar has an outer perimeter covered with an insulative material. In another option, the outer surface of the coiled conductor 130 includes one or more layers of coating of insulating material thereon, or an insulative sleeve. Examples of materials suitable for an insulative coating or an insulative sleeve disposed on the coil conductors 130 includes, but is not limited to PTFE, EFTE, or polyurethane, or materials capable of providing electrical isolation, abrasion resistance and reduced friction. The insulative material allows for the one or more cable conductors 124 to rest directly again the coated or insulated coil conductor 130, while the remainder of the radial space about the coil conductors being taken by one or more fillers 140, as further discussed below. The insulative material promotes isolation, and improves electrical isolation, without giving up extendable/retractable functionality of a design that includes such features.

The coiled conductor 130 forms a lumen 132 (FIG. 3) therein. The lumen 132 (FIG. 3) allows for instruments to be disposed therein, such as a stylet or a guidewire, facilitating placement or manipulation of the lead with the patient. The coiled conductor 130, in one option, is not aligned with a longitudinal axis 108 (FIG. 4) of the lead body 110. For example, the coiled conductor 130, in one option, is offset from the longitudinal axis 108 of the lead body 110, as illustrated in FIG. 3.

The lead body 110 further includes one or more fillers 140 disposed therein. The one or more fillers 140 are each an elongate structure that are disposed within the lead body 110, and in option are formed of silicone. The one or more fillers 140 serve to fill the remaining cross-sectional area of the lead body 110 that the conductors do not fill. In one option, the one or more fillers 140 fill about 50% or less than the cross-sectional area of the lead body 110.

In one option, two fillers 140 are disposed within the lead body 110. In another option, the one or more fillers 140 serve to fill the lead body 110, but do not require the conductors to be disposed within recesses of the filler, or otherwise coupling the conductors with the filler. The one or more fillers 140, in another option, have an overall C-shape, allowing the one or more fillers 140 to conform to a part of the inner surface of the lead body 110. The one or more fillers 140 are, in one option, flexible with optional various stiffnesses. In one option, the one or more fillers 140 are more flexible than the lead body 110.

The one or more fillers 140, in one option, include compression features, such as having a wave-like shape 141 on all or a portion of its perimeter, allowing the fillers to compress within the lead body 110, or otherwise move with the lead body 110, for example when the lead 100 is manipulated within the vasculature of a patient. In one option, the compression features are formed on an inner perimeter of the fillers 140. Other features other than a wave-like shape can be incorporated with the fillers for the compressions features, such as, but not limited to features that disrupt the outer perimeter, or sawtooth features. As mentioned above, the one or more fillers 140 serve to fill the lead body 110. Having two or more fillers 140, allows for increased number of options in the design of the lead. For example, in one option, two fillers are disposed on opposite sides of the coiled conductor, as illustrated in FIG. 2. In another option, as illustrated in FIG. 3, one filler 140 having a generally C-shape is disposed within the lead body 110. The C-shape has a first end 148 and a second end 149, and an inner perimeter portion 147. The coiled conductor 130 is positioned within the filler, as shown, but is not necessarily coupled with the filler 140. One or more cable conductors 124, for example, two cable conductors 124, are disposed between the two ends 142 of the filler 140, for example, the two first ends 148.

In yet another example, three cable conductors 124 are disposed within the lead body 110, as illustrated in FIG. 4. The fillers 140 have, in one option, a variety of sizes disposed within the lead body 110, for example, as shown in FIG. 4. The variations in filler 140 size also allows for having an asymmetrical arrangement of cables and coil conductors, providing further options for decreasing the overall amount of filler needed, and decreasing the overall size of the lead body 110.

A method is further included, that incorporates that variations discussed above, as well as further options. The method includes disposing two or more conductors within an insulative lead body, where the two or more conductors include a coiled conductor and at least one cable conductor. The method further includes electrically coupling an electrode with at least one conductor, and disposing one or more fillers within the lead body without coupling the conductors with the one or more fillers.

Several options for the method are as follows. For example, in one option, disposing the coiled conductor within the lead body includes disposing the coiled conductor at a location offset from a longitudinal axis of the lead body. In another option, disposing one or more fillers includes disposing two or more fillers on opposite sides of the coiled conductor. The method further includes, in another option, insulating the coiled conductor and the at least one cable conductor. The coiled conductor and/or the cable conductor can be independently insulated. In another option, the coiled conductor and/or the cable conductor can include one or more layers of insulation, such as a coating, or a sleeve of material. In yet another option, disposing one or more fillers within the lead body includes disposing a C-shaped filler within the lead body, the C-shape having an inner perimeter portion, and disposing the coiled conductor within the inner perimeter portion. In another embodiment, disposing one or more fillers within the lead body includes disposing one or more fillers with compression features within the lead body.

The assembly can be created by coating or sheathing the inner members with an outer insulative layer, and prevent the need for stringing conductors through small lumens. The filler, optionally extruded filler, has controllable compressibility and stiffness, while accommodating various conductor sizes, quantity, and placement, without compromising the ability to use a stylet or extendable designs. The filler allows for an efficient use of volume within the outer sleeve, allowing for a smaller sleeve to be used.

It is to be understood that the above description is intended to be illustrative, and not restrictive. Although the use of the implantable device has been described for use as a lead in, for example, a cardiac stimulation system, the implantable device could as well be applied to other types of body stimulating systems. Many other embodiments will be apparent to those of skill in the art upon reviewing the above description. The scope should, therefore, be determined with reference to the appended claims, along with the full scope of equivalents to which such claims are entitled.

1. An implantable lead comprising:
a lead body;
at least one electrode disposed along the lead body;
at least one conductor electrically coupled with the at least one electrode;
one or more fillers disposed within the lead body, the one or more fillers are disposed adjacent to the at least one conductor, but are not coupled with the conductor.

2. The implantable lead as recited in claim 1, wherein the one or more fillers include compression features associated therewith.

3. The implantable lead as recited in claim 2, wherein the compression features include compression waves disposed on an inner perimeter of the one or more fillers.

4. The implantable lead as recited in claim 1, wherein two fillers are disposed within the lead body, each filler having a first end and a second end, and a first conductor is disposed between two first ends of the two fillers, and a second conductor is disposed between two second ends of the two fillers.

5. The implantable lead as recited in claim 1, further comprising a coiled conductor forming a lumen therein, the coiled conductor disposed within the lead body, and a coil conductor longitudinal axis is offset from a lead body longitudinal axis.

6. The implantable lead as recited in claim 1, wherein the one or more fillers is generally C-shaped.

7. The implantable lead as recited in claim 1, wherein the one or more fillers is formed of silicone.

8. An implantable lead comprising:
an elongate lead body, the elongate lead body having a cross-sectional area;
at least one electrode disposed along the elongate lead body;
at least one conductor disposed within the elongate lead body;
one or more fillers disposed within the lead body, each filler filling less than about 50% of the cross-sectional area.

9. The implantable lead as recited in claim 8, wherein two fillers are disposed within the lead body.

10. The implantable lead as recited in claim 9, wherein each filler is generally C-shaped.

11. The implantable lead as recited in claim 9, wherein each filler includes compression features associated therewith.

12. The implantable lead as recited in claim 10, wherein the filler extends from a first end to a second end and having an inner perimeter therein, and an insulated coiled conductor is disposed within the C-shape and adjacent the inner perimeter.

13. The implantable lead as recited in claim 12, further comprising at least one insulated cable conductor disposed between the first end and the second end.

14. The implantable lead as recited in claim 13, wherein the at least one insulated cable conductor includes two cable conductors disposed directly adjacent to one another and between the first and second ends.

15. A method comprising:
disposing two or more conductors within an insulative lead body, where the two or more conductors include a coiled conductor and at least one cable conductor;
electrically coupling an electrode with at least one conductor; and
disposing one or more fillers within the lead body without coupling the conductors with the one or more fillers.

16. The method as recited in claim 15, wherein disposing the coiled conductor within the lead body includes disposing the coiled conductor at a location offset from a longitudinal axis of the lead body.

17. The method as recited in claim 15, wherein disposing one or more fillers includes disposing two or more fillers on opposite sides of the coiled conductor.

18. The method as recited in claim 15, further comprising insulating the coiled conductor and the at least one cable conductor with one or more layers of insulation.

19. The method as recited in claim 15, wherein disposing one or more fillers within the lead body includes disposing a C-shaped filler within the lead body, the C-shape having an inner perimeter portion, and disposing the coiled conductor within the inner perimeter portion.

20. The method as recited in claim 15, wherein disposing one or more fillers within the lead body includes disposing one or more fillers with compression features within the lead body.
